(19) United States
(12) Patent Application Publication
(10) Pub. No.: US 2009/0041745 A1
(43) Pub. Date: Feb. 12, 2009

(54) MODULATORS OF PARAPTOSIS AND RELATED METHODS

(76) Inventors: Sabina SPERANDIO, Novato, CA (US); Dale E. Bredesen, Novato, CA (US)

Correspondence Address: MORRISON & FOERSTER LLP, 755 PAGE MILL RD, PALO ALTO, CA, 94304-1018, US

(21) Appl. No.: 12/167,912

(22) Filed: Jul. 3, 2008

Related U.S. Application Data
(63) Continuation of application No. 10/079,929, filed on Feb. 19, 2002, now abandoned.

(51) Int. Cl. A61K 38/48; C12N 5/06; A61P 9/00; A61P 25/00; A61K 38/00

(52) U.S. Cl. 424/94.63; 435/375; 514/12

(57) ABSTRACT

The invention is directed to a method of modulating paraptotic cell death in a cell by contacting the cell with an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, wherein the effective amount of the compound induces paraptotic death of the cell. The invention further is directed to a method of inhibiting paraptotic cell death in a cell by contacting the cell with an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein the effective amount of the compound inhibits paraptotic death of the cell.

This invention was made with government support under grant number AG12282 awarded by the National Institutes of Health. The United States Government has certain rights in this invention.

### BACKGROUND OF THE INVENTION

The present invention relates generally to molecular medicine and programmed cell death and more specifically to methods of modulating non-apoptotic programmed cell death. Apoptosis is the most common and best understood of the programs of cell death. The central set of cysteine-aspartyl proteases or caspases that drive the process are instrumental in the vast majority of apoptotic events that occur during normal embryonic development, as was initially illustrated in studies of the nematode Caenorhabditis elegans where elimination of the caspase homologue led to complete cessation of the 131 programmatic cell deaths that normally occur during development of that organism. The role of caspase-driven apoptotic events in human pathogenesis is less clear. However, recent evidence supports the theory that caspase cleavage of mutant proteins may represent an important signaling event in the initiation of cell death in a variety of degenerative conditions.

Despite the widespread occurrence of apoptosis in physiological and pathological cell death, the occurrence of cell deaths that fulfill criteria for neither apoptosis nor necrosis has been well documented.

For example, certain developmental cell deaths, such as autophagic cell death and cytoplasmic cell death, do not resemble apoptosis. Furthermore, neurodegenerative diseases such as Huntington's disease and amyotrophic lateral sclerosis are characterized by neuronal cell death that is nonapoptotic. In addition, ischemia-induced cell deaths may also display a non-apoptotic morphology, referred to as “oncosis.” The biochemical mechanisms involved in these alternative forms of cell death remain largely unknown. However, discovery of their existence means that modulation of the apoptotic pathway genetically or pharmacologically may prove ineffective in situations in which such nonapoptotic cell death occurs.

One form of programmed cell death that is distinct from apoptosis by the criteria of morphology, biochemistry and response to apoptosis inhibitors has been termed “paraptosis.” Despite its lack of response to caspase inhibitors and Bcl-X_{L}, paraptotic cell death has been shown to be induced, among other inducers, by insulin-like growth factor I receptor (IGFIR) and mediated by an alternative caspase-9 activity that is Apaf-1 independent.

Nonapoptotic cell death has been implicated in developmental cell death, neurodegenerative diseases and cancer. Thus, a need exists to identify compounds that modulate paraptosis and develop methods for both the induction and inhibition of paraptosis. The present invention satisfies this need and provides related advantages as well.

### SUMMARY OF THE INVENTION

The invention is directed to a method of inducing paraptotic cell death in a cell by contacting the cell with an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, wherein the effective amount of the compound induces paraptotic death of the cell. The invention further is directed to a method of inhibiting paraptotic cell death in a cell by contacting the cell with an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein the effective amount of the compound inhibits paraptotic death of the cell.

Also provided by the invention is a method of treating a condition associated with excessive cell accumulation by administering to a subject in need of such treatment an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, wherein the effective amount of the compound induces paraptotic cell death. The invention further provides a method of treating a condition associated with excessive cell death by administering to a subject in need of such treatment an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein the effective amount of the compound inhibits paraptotic cell death.

### BRIEF DESCRIPTION OF THE DRAWINGS

FIG. 1 shows a schematic that demonstrates that paraptosis and apoptosis together represent a two-pronged response to death-inducing insults.

FIG. 2 shows a diagram demonstrating differential gene expression in paraptosis and apoptosis, with co-expression of only 2 of 116 transcripts.

FIG. 3 shows a table setting forth the lack of effect on paraptotic cell death of various apoptosis inhibitors.

FIG. 4 shows a table setting forth morphological differences observed between apoptosis and paraptosis.

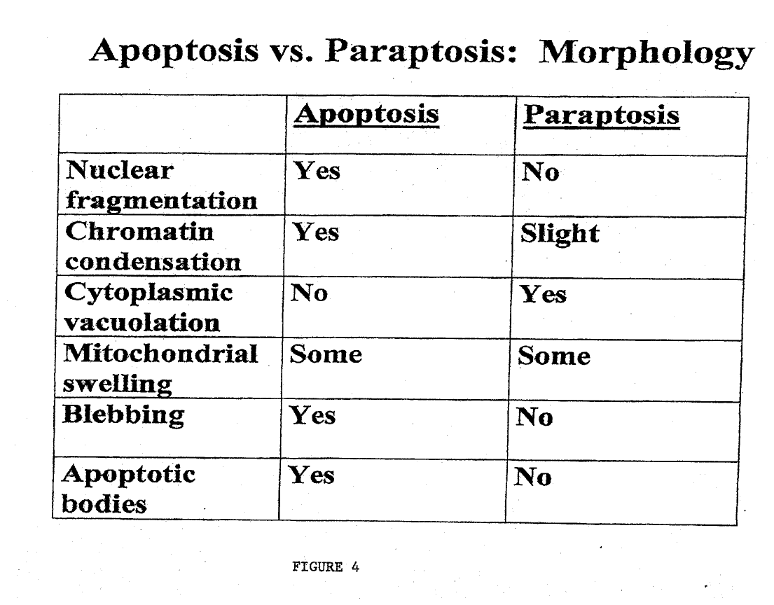

FIG. 5 shows a table setting forth biochemical distinctions observed between apoptosis and paraptosis.

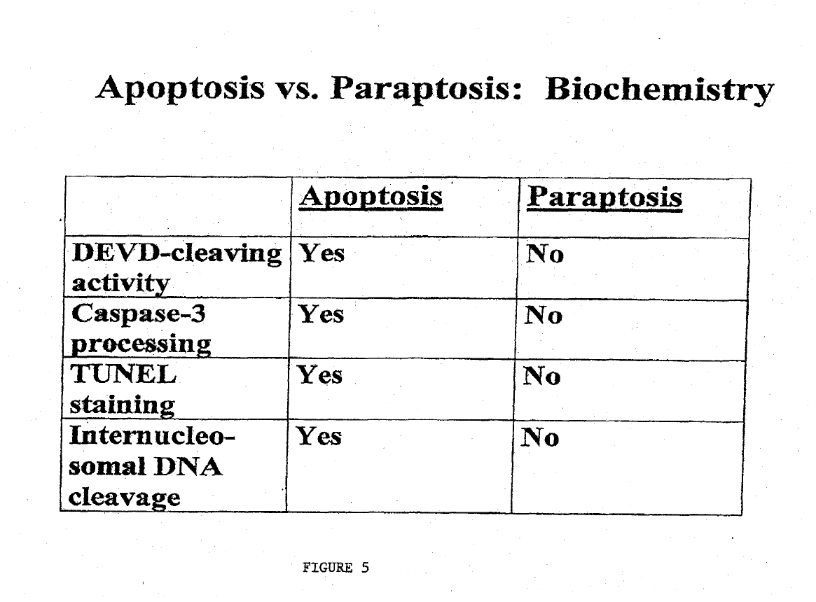

### DETAILED DESCRIPTION OF THE INVENTION

The invention is directed to methods of modulating paraptosis with compounds that have the ability either to induce or to inhibit this particular form of programmed cell death. Paraptosis, a form of programmed cell death, is implicated in conditions involving cell death-inducing insults as well as in conditions associated with the inhibition of cell death. Therefore, compounds that modulate paraptosis, can be used therapeutically in the treatment of a variety of conditions including neoplastic conditions, autoimmune conditions, neurodegenerative conditions and ischemic conditions.

The methods of the invention for modulating paraptosis have therapeutic value for a variety of conditions associated with aberrant levels of paraptosis. For example, the invention methods can be used to treat those types of neurodegeneration associated with nonapoptotic cell death such as, for example, familial amyotrophic lateral sclerosis as described by Dal Canto and Gurney, American Journal of Pathololoqy 145:1271-1279 (1994); Huntington's Disease as described by Turmaine et al., Procl. Nat. Acad. Sci. USA 97:8093-8097 (2000). Notably, there is no evidence that the majority of neural cell death in other neurodegenerative diseases including Parkinson's Disease and Alzheimer's Disease is apoptotic in nature.

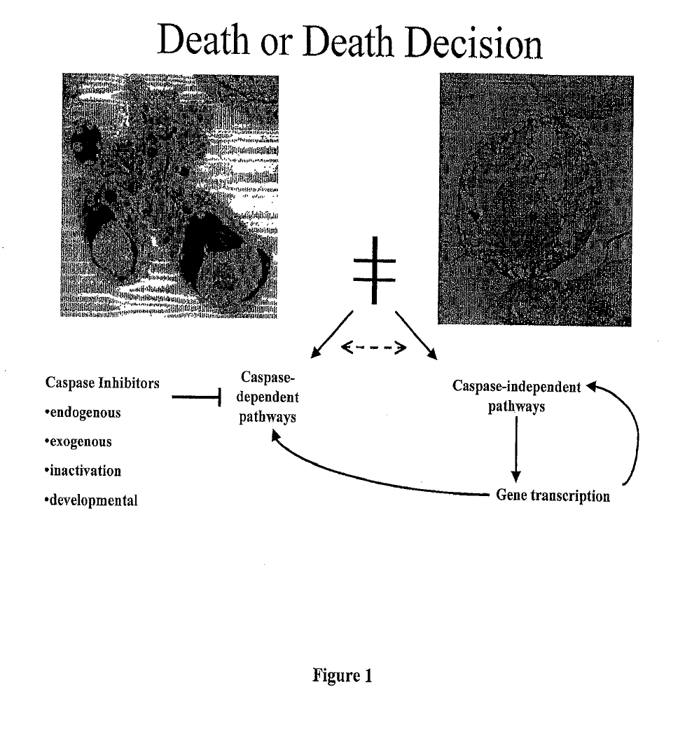

As shown in FIG. 1, cell-death inducing insults can lead to a two-pronged response, where one arm of the pathway is caspase-dependent and leads to apoptotic cell death, while a separate arm that is caspase independent leads to paraptotic cell death. Receptors involved in mediating cell death may activate either the paraptotic or apoptotic pathway, or may activate both pathways. Receptors that activate the paraptotic pathway, for example, Insulin-Like Growth Factor I Receptor (IGFIR), typically inhibit caspases. Receptors that can activate both pathways, for example, the TNF-α receptor, typically respond to caspase inhibition by switching from activation of the apoptotic pathway to activation of the paraptotic pathway.

Whether cell death occurs via paraptosis or apoptosis depends on a variety of factors, including, for example, the cell type and the type of insult. In particular, if a cell is contacted with a toxin such as, for example, a sulthydryl oxidizing agent such as diethylmaleate, and damage to the endogenous caspases results in their inactivation, then cell death will occur via the paraptotic pathway. Similarly, if the cell that is subjected to the insult is producing an endogenous caspase inhibitor, for example, xiap, apoptosis also is blocked in favor of paraptosis. Gene expression from the non-caspase cell death pathway leads to upregulation of both pro-paraptotic and pro-apoptotic molecules. As described herein, inhibitors or neutralizing agents of the Jun N-terminal kinases (JNKs) JNK1 and JNK2, which are MAP kinases activated in response to cellular stress, block both the paraptotic and the apoptotic cell death pathways. Furthermore as described herein, Tumor Necrosis Factor (TNF) and ceramide are two compounds that can induce paraptosis.

Cross-talk between the paraptotic and apoptotic pathways is a further feature of programmed cell death. For example, caspase-9 can induce the paraptotic pathway in an Apaf-1-independent manner as described in Sperandio et al., Procl. Natl. Acad. Sci. USA 97:14376-14381 (2000), which is incorporated herein by reference. In addition and as described herein, caspase-7 and caspase-8 also can induce the paraptotic pathway, but unlike for caspase-9, induction by these two caspases is subject to inhibition by, for example, zVAD.fmk, BAF amd p35. In addition, caspase-7 inhibitors include, for example, xiap and Ac-DEVD, and caspase-8 inhibitors include, for example, IETD.fmk and crmA.

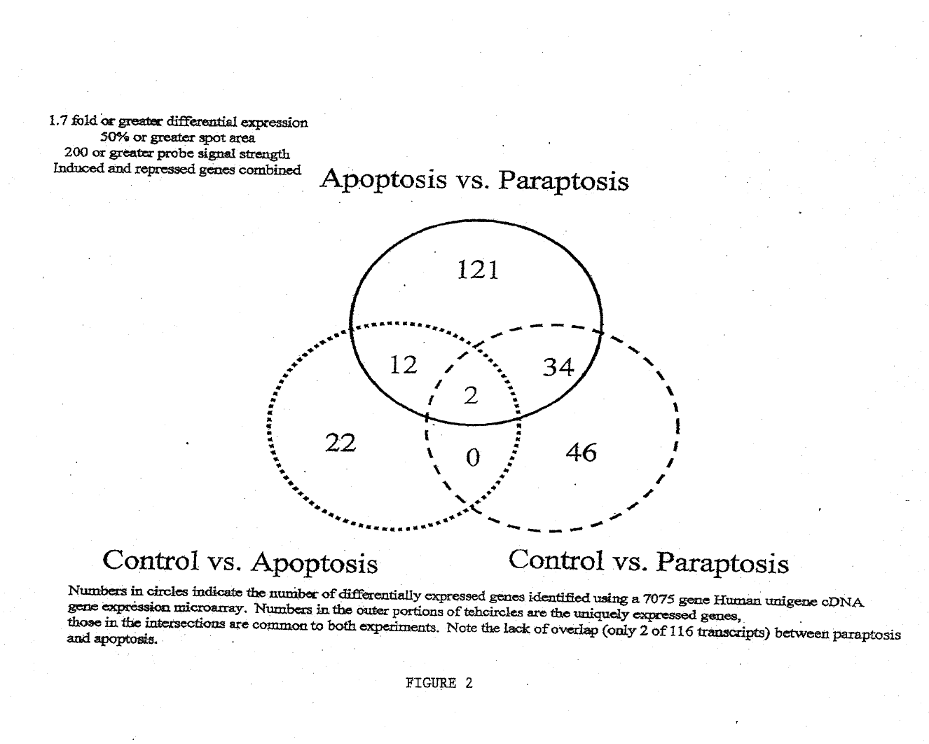

Insulin-Like Growth Factor I Receptor (IGFIR) as well as the IGFIR intracytoplasmic domain (IGFIR-IC), are paraptosis-mediating molecules that induce a form of non-apoptotic programmed cell death characterized by cytoplasmic vacuolation and resistance to apoptosis inhibitors. This form of cell death, designated paraptosis, requires transcription and de novo protein synthesis. In addition, a microarray screening comparing gene expression profiles between IGFIR induced cell death and apoptotic cell death showed that fewer than 2% of those genes that are differentially expressed are shared between the two cell death programs, a finding consistent with distinct cell death programs. As shown in FIG. 2, a Human unigene microarray of 7075 genes shows an overlap of only 2 of 116 transcripts between paraptosis and apoptosis.

Paraptotic cell death has been implicated in a variety of pathological and normal cellular events.

Overexpression of fragments of the intracellular domain of IGFIR in cancer cells has been shown to reduce tumorigenicity in nude mice and induce cell death (see, Hongo et al., Cancer Research 58:2477-2484 (1998); Liu et al., Cancer Research 58:570-576 (1998)). The expression of IGFIR is decreased in prostate cancer (Tennant et al., J. Clin. Endocrinol. Metab. 81:3774-82 (1996)₁, and its reexpression in immortalized human prostate cells inhibited the malignant phenotype (Plymate et al., Endocrine 7:119-24 (1997)). Moreover, a potential role for IGFIR in developmental cell death is suggested by the phenotype of IGFIR-null mice, which includes a higher neuronal density in the brain stem and spinal cord (Liu et al. Cell 75:59-72 (1993)). The paraptotic form of programmed cell death induced by IGFIR and IGFIR-IC has been shown to be distinct from apoptosis based on morphological, biochemical and molecular features as described in Sperandio et al., supra, 2000, which is incorporated herein by reference in its entirety.

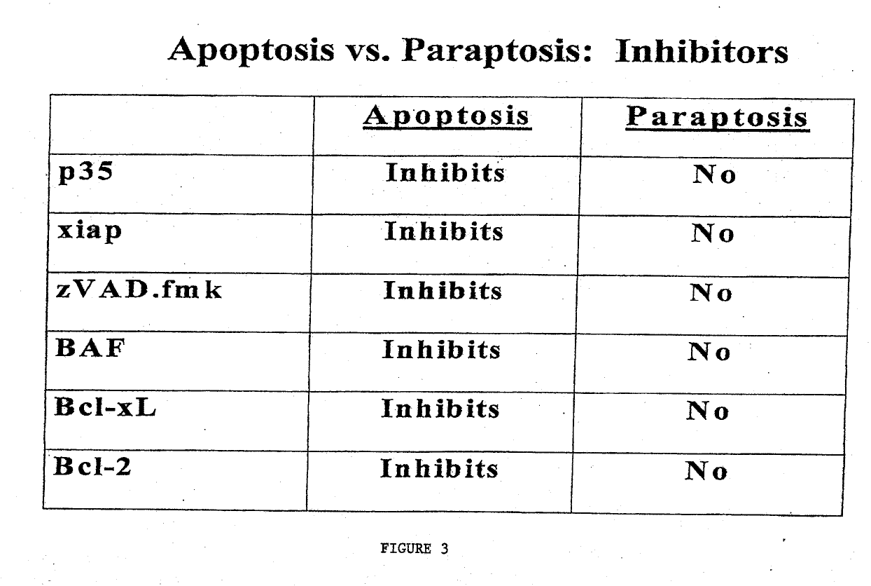

Paraptosis is a nonapoptotic form of programmed cell death that can be induced by IGFIR and is mediated by a newly identified function of caspase-9 that is distinct from the role of caspase-9 in the apoptotic pathway as described in Sperandio et al., supra, 2000. Thus, caspase-9 participates in both, apoptotic and paraptotic forms of cell death. However, as shown in FIG. 3, caspase inhibitors that inhibit apoptosis fail to inhibit paraptosis, an indication that distinct catalytic activities of caspase-9 mediate apoptosis and paraptosis. IGFIR co-imminoprecipitates with caspase-9 and mutants of IGFIR that fail to coimmunoprecipitate with caspase-9 also fail to induce paraptosis. The pro-apoptotic and the pro-paraptotic effects of caspase-9 can be distinguished by the lack of effect of caspase inhibitors including, for example, BAF, zVAD.fmk, p35, and xiap on paraptosis; lack of a requirement of paraptosis for activation of caspase-9 zymogen by Apaf-1; and lack of suppression of paraptosis by mutation catalytic sites of caspase-9 as described in Sperandio et al., supra, 2000. Thus, caspase-9 has at least two distinct activities, one that is pro-apoptotic and one that induces paraptotic cell death.

In one embodiment, the invention provides a method of inducing paraptotic cell death in a cell by contacting the cell with an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, where the effective amount of the compound induces paraptotic cell death

In a further embodiment, the invention provides a method of inhibiting paraptotic cell death in a cell by contacting the cell with an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein the effective amount of the compound inhibits paraptotic death of the cell.

As used herein, the term “paraptotic cell death” refers to programmed non-apoptotic cell death that can be mediated by caspase-9 and lacks many of the molecular, biochemical and cytological characteristics of apoptosis. Paraptosis and apoptosis represent two separate programs of cell death that are induced via distinct molecular pathways, but may be induced simultaneously by a single insult or agent. One feature distinguishing paraptotic cell death from apoptotic cell death is that paraptotic cell death is not affected by caspase inhibitors. In particular, inhibition of apoptosis by the caspase inhibitors zVAD.fmk, BAF, p35m, X-chromosome-linked inhibitor of apoptosis (xiap), Bcl-2, and Bcl-2 family member Bcl-xL are features associated with apoptosis that are not observed or greatly reduced in non-apoptotic cell death. A further distinction between paraptotic cell death and apoptotic cell death is the dependence of apoptotic cell death on Apaf-1, the cytosolic cofactor of caspase-9 zymogen. The mediation of paraptotic ccl death when caspase-9 is a mediator is a function of caspase-9 that is distinct from the role of caspase-9 in the apoptotic pathway as evidenced, for example, by the Apaf-1 independence of the paraptotic pathway.

With regard to morphological distinctions between paraptotic and apoptotic cell death, nuclear fragmentation, apoptotic body formation and chromatin condensation are ultrastrucural features of apoptosis that are not observed or are greatly reduced in paraptosis. Furthermore, paraptosis can be associated with cytoplasmic vacuolation, a feature not observed in apoptosis. In addition to being distinct from apoptotic cell death, paraptosis is further distinct from non-programmed cell death, known as necrosis. For example, cleavage of poly(ADP-ribose)polymerase (PARP) yielding distinctive 50 to 62 kDa fragments is a feature of necrosis that is not observed in nonapoptic cell death. Thus, paraptotic cell death is a non-apoptotic form of programmed cell death.

As used herein, the phrase “in a cell” is intended to mean within a living organism or living cell. A living organism includes for example, multi-cellular organisms such as a human, animal, insect, or worm, and uni-cellular organisms such as a single-celled protozoan, yeast cell, or bacterium. In addition, a living cell derived from an organism used directly or grown in cell culture is an in vivo environment that also is encompassed by the phrase “in a cell.” For example, an oocyte removed from an organism such as a frog used directly or grown in a tissue culture dish would constitute an in vivo environment encompassed by the phrase “in a cell.”

As used herein, the term “neutralizing agent” is intended to refer to an agent effecting a decrease in the activity, amount or rate of expression of the reference molecule or compound, for example, Jun N-terminal kinase 1 (JNK1) or JNK2.

Neutralizing agents useful for practicing the claimed invention include, for example, binding molecules such as antibodies as well as molecules that modulate or regulate the activity, amount or rate of expression of the reference molecule or compound through non-binding interactions. A neutralizing agent can be, for example, any molecule that binds JNK1, JNK2, TRAF2 or any other reference molecule with sufficient affinity to decrease its activity. Additionally, a neutralizing agent can be any molecule binds to a regulatory molecule or gene region so as to inhibit or promote the function of the regulatory protein or gene region and effect a decrease in the extent or amount or rate of expression or activity of JNK1, JNK2, TRAF2 or any other reference molecule. Thus, a neutralizing agent can be any molecule that directly or indirectly modulates or regulates the extent, amount or rate of expression or activity of JNK1, JNK2, TRAF2 or any other reference molecule. For example, a peptide or peptidomimetic that binds JNK1, JNK2, TRAF2 or any other reference molecule with sufficient affinity to decrease activity, respectively, is useful for practicing the claimed methods. In addition, examples of neutralizing agents which effect a decrease in the expression of JNK1, JNK2, TRAF2 or any other reference molecule can include antisense nucleic acids and transcriptional inhibitors.

As used herein, the term “effective amount” when used in reference to a compound or molecule that is an inhibitor or inducer of paraptotic cell death, is intended to mean an amount of the compound or molecule sufficient to treat or reduce the severity of a condition in an affected subject.

A compound useful for practicing the invention method for modulating paraptosis, a term used herein to refer collectively to the processes of inducing and inhibiting paraptosis, can act through a variety of mechanisms, for example, by altering the association of IGFIR and caspase-9 in a population of cells and, therefore, can be useful as medicaments for treating a pathology characterized by an aberrant level of paraptosis. Such a compound can, for example, decrease the affinity of association of IGFIR and caspase-9.

Paraptosis can be induced, for example, by upregulating caspase-9 in the presence of an apoptosis inhibitor. In this regard, the expression of caspase-9 in the presence of the apoptosis inhibitor BAF (or zVAD.fmk) induces paraptosis as described in Sperandio et al., supra, 2000. Thus, paraptosis can be induced by upregulating caspase-9 in the presence of an apoptosis inhibitor. Similarly, upregulation of caspase-2, caspase-7 or caspase-8, will induce both paraptosis and apoptosis. As shown herein, paraptosis also can be induced by, for example, ceramide, Tumor Necrosis Factor (TNF), caspase-7, caspase-8, α-amino-3-hydroxy-5-methyl-4-isoxazole proprionic acid (AMPA), kainic acid and glutamic acid. In addition, paraptosis can be induced by an environmental stimulus that represents a cell death-inducing insult such as, for example, heatshock, and the like.

Once paraptosis has been induced in a cellular system, a compound capable of modulating paraptosis such as, for example, the compounds administered in the methods of the present invention, can be identified by contacting the cellular system with a test-compound to verify that paraptosis is modulated. One skilled in the art will appreciate that further compounds having the same or similar modulating activities as the compounds administered in the invention methods can be identified from among a diverse population of molecules. Methods for producing libraries containing diverse populations of molecules, including chemical or biological molecules such as simple or complex organic molecules, peptides, proteins, peptidomimetics, glycoproteins, lipoproteins, polynucleotides, and the like, are well known in the art (Huse, U.S. Pat. No. 5,264,563, issued Nov. 23, 1993; Blondelle et al., Trends Anal. Chem. 14:83-92 (1995); York et al., Science 274:1520-1522 (1996); Gold et al., Proc. Natl. Acad. Sci. USA 94:59-64 (1997); Gold, U.S. Pat. No. 5,270,163, issued Dec. 14, 1993). Such libraries also can be obtained from commercial sources.

Since libraries of diverse molecules can contain as many as 10¹⁴ to 10¹⁵ different molecules, a screening assay provides a simple means for identifying further compounds that can modulate paraptosis. In particular, a screening assay can be automated, which allows for high through-put screening of randomly designed libraries of compounds to identify further compounds that can modulate paraptosis.

Modulation of paraptosis using the methods of the invention can be a therapeutic strategy for treatment of a variety of neurodegenerative conditions, ischemic conditions, autoimmune conditions as well as neoplastic conditions as set forth herein. As such, administration of a compound that inhibits paraptosis can lead to a reduction in the severity of an ischemic condition, neurodegenerative condition or any other condition that is associated with increased cell death. Neural cell death diseases include neurodegenerative diseases such as retinal degeneration, Huntingtons Disease, Parkinson's Disease and Alzheimer's Disease as well as other diseases associated with the loss of neural cells including, for example, stroke, trauma, global ischemia, hypoxia, seizure-induced excitotoxicity. Exemplary compounds contemplated for inhibiting paraptosis include paraptosis-modulating compounds administered in the invention methods and, for example, Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125.

Administration of a paraptosis-modulating compound that induces paraptosis such as, for example, ceramide, Tumor Necrosis Factor (TNF), caspase-7, caspase-8, α-amino-3-hydroxy-5-methyl-4-isoxazole proprionic acid (AMPA), kainic acid and glutamic acid, can lead to a reduction in the severity of a neoplastic condition or autoimmune condition or any other condition associated with excessive cell accumulation. A “neoplastic condition,” as used herein, refers to a condition associated with hyperproliferation of cells and includes benign and malignant expanding lesions of proliferating cells. A neoplastic condition is thus characterized by a reduction or deceleration in cell death resulting from a loss of homeostatic control of the appropriate number of cells in a normal tissue. A benign neoplasm grows in an expansile manner, displacing or compressing surrounding tissues rather than invading them. A malignant neoplasm or cancer, refers to a large group of diseases characterized by uncontrolled growth and spread of abnormal cells and includes any condition of tumors having the properties of anaplasia, invasion, and metastasis.

A paraptosis-modulating compound can be a compound or molecule that binds IGFIR or another paraptosis-mediating molecule with sufficient affinity to modulate paraptosis. One skilled in the art will appreciate that in addition to the compounds taught herein to be modulators of paraptosis additional paraptosis-modulating compounds can be identified and can be, for example, a macromolecule, such as polypeptide, nucleic acid, carbohydrate or lipid. Thus, a paraptosis-modulating compound can be an antibody, antisense nucleic acid and any compound identified by the methods herein and known to those skilled in the art. A paraptosis-modulating compound can also be a derivative, analogue or mimetic compound as well as a small organic compound as long as paraptosis is modulated in the presence of the compound. The size of a paraptosis-modulating compound is not important so long as the molecule exhibits or can be made to exhibit paraptosis-modulating activity. For example, a paraptosis-modulating compound can be as little as between about one and six, and as large as tens or hundreds of monomer building blocks which constitute a macromolecule or chemical binding molecule. Similarly, an organic compound can be a simple or complex structure so long as it has sufficient paraptosis-modulating activity.

Paraptosis-modulating compounds useful for practicing the methods of the invention include the paraptosis-inducing compounds ceramide, Tumor Necrosis Factor (TNF), caspase-7, caspase-8, α-amino-3-hydroxy-5-methyl-4-isoxazole proprionic acid (AMPA), kainic acid and glutamic acid; and further include the paraptosis-inhibiting compounds Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125. In addition to the specific paraptosis-modulating compounds taught herein, paraptosis-modulating compounds also can include, for example, antibodies and other receptor or ligand binding polypeptides of the a immune system. Such other molecules of the immune system include for example, T cell receptors (TCR) including CD4 cell receptors. Additionally, cell surface receptors such as integrins, growth factor receptors and chemokine receptors, as well as any other receptors or fragments thereof that bind to an endogenous paraptosis-mediating molecule such as, for example, IGFIR or caspase-9, or can be made to bind to an endogenous paraptosis-mediating molecule, with sufficient affinity to modulate activity are also paraptosis-modulating compounds useful for practicing the methods of the invention. Examples of selective inhibitors of paraptosis include, for example, Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline, the JNK inhibitor SP 600125, IGFIR neutralizing agents and transcriptional inhibitors that bind to the IGFIR promoter/regulatory region. Additionally, receptors, ligands, growth factors, cytokines or chemokines, for example, which inhibit the expression of an endogenous paraptosis-mediating molecule are also paraptosis-modulating compounds useful for practicing the methods of the invention. Furthermore, DNA binding polypeptides such as transcription factors and DNA replication factors are likewise included within the definition of the term binding molecule so long as they have selective paraptosis-modulating activity. Finally, polypeptides, nucleic acids and chemical compounds such as those selected from random and combinational libraries can also be paraptosis-modulating compounds.

Various approaches can be used for identifying further paraptosis-modulating compounds useful for practicing the invention methods. For example, a paraptosis-modulating compound that inhibits paraptosis can be an antibody and other receptor of the immune repertoire that acts as a neutralizing agent for, for example, Jun N-terminal kinase 1 (JNK1), Jun N-terminal kinase 2 (JNK2), TNF Receptor-Associated Factor 2 (TRAF2). Therefore, generating a diverse population of binding molecules from an immune repertoire, for example, can be useful for identifying further paraptosis-modulating compounds in addition to those taught and exemplified herein.

A further paraptosis-modulating compound useful for practicing the invention methods can also be identified from a large population of unknown molecules by methods well known in the art. Such a population can be a random library of peptides or small molecule compounds. The population can be generated to contain a sufficient diversity of sequence or structure so as to contain a molecule which will bind to an endogenous paraptosis-mediating molecule such as, for example, IGFIR, caspase-7, caspase-8 or caspase-9, or their respective nucleic acids. Those skilled in the art will know what size and diversity is necessary or sufficient for the intended purpose. A population of sufficient size and complexity can be generated so as to have a high probability of containing a paraptosis-modulating compound that binds an endogenous paraptosis-mediating molecule such as, for example, IGFIR, caspase-7, caspase-8 or caspase-9, with sufficient affinity to modulate activity. Numerous other types of library molecule populations exist and are described further below.

Any molecule that binds to an endogenous paraptosis-mediating molecule, to a gene region that controls expression of such a molecule, or to a regulatory molecule that modulates activity or expression of an endogenous paraptosis-mediating molecule, as well as to any regulatory molecule that modulates IGFIR expression is a paraptosis-modulating compound useful for practicing the invention. For example, a paraptosis-modulating compound can be a regulatory molecule affects an expression of an endogenous paraptosis-mediating molecule such as, for example, IGFIR, caspase-9, caspase-7, or caspase-8, by modulating the action of a transcription factor that controls or upregulates transcription of the endogenous paraptosis-mediating molecule. In addition, a regulatory molecule that binds with sufficient affinity to a molecule involved in the activation of an endogenous paraptosis-mediating molecule to reduce paraptosis is a paraptosis-modulating compound useful for practicing the methods of the invention.

A moderate sized population for identification of a further paraptosis-modulating compound useful for practicing the methods of the invention can consist of hundreds and thousands of different binding molecules within the population, whereas a large sized binding molecule population will consist of tens of thousands and millions of different binding molecule species. More specifically, large and diverse populations of binding molecules for the identification of a further paraptosis-modulating compound will contain any of about 10⁴, 10⁵, 10⁶, 10⁷, 10⁸, 10⁹, 10¹⁰, or more, different molecule species. One skilled in the art will know the approximate diversity of the population of test-compounds sufficient to identify additional paraptosis-modulating compounds useful for practicing the methods of the invention.

A paraptosis-modulating compound useful for practicing the invention method can also be identified by using purified polypeptide to produce antibodies, which can serve as neutralizing agents of the invention. For example, antibodies which are specific for caspase-7, caspase-8, caspase-9, JNK1, JNK2 or TRAF2, or another endogenous paraptosis-mediating compound can be used as a paraptosis-modulating compound of the invention and can be generated using methods that are well known in the art. Such paraptosis-modulating compounds can include both polyclonal and monoclonal antibodies against IGFIR, caspase-7, caspase-8, caspase-9, JNK1, JNK2 or TRAF2, or any endogenous paraptosis-mediating molecule, as well as antigen binding fragments of such antibodies including Fab, F(ab′)2, Fd and Fv fragments and the like. In addition, further paraptosis-modulating compounds useful for practicing the methods of the invention encompass non-naturally occurring antibodies, including, for example, single chain antibodies, chimeric antibodies, bifunctional antibodies, complementarity determining region-grafted (CDR-grafted) antibodies and humanized antibodies, as well as antigen-binding fragments thereof.

Methods of preparing and isolating antibodies, including polyclonal and monoclonal antibodies, using peptide immunogens, are well known to those skilled in the art and are described, for example, in Harlow and Lane, Antibodies: A Laboratory Manual, Cold Spring Harbor Laboratory Press (1988). Non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (19891, which is incorporated herein by reference. These and other methods of making, for example, chimeric, humanized, CDR-grafted, single chain, and bifunctional antibodies are well known to those skilled in the art (Hoogenboom et al., U.S. Pat. No. 5,564,332, issued Oct. 15, 1996; Winter and Harris, Immunol. Today 14:243-246 (1993); Ward et al., Nature 341:544-546 (1989); Harlow and Lane, Antibodies: A Laboratory Manual (Cold Spring Harbor Laboratory Press, 1988); Hilyard et al., Protein Engineering: A Practical Approach (IRL Press 1992); Borrabeck, Antibody Engineering, 2d ed. (Oxford University Press 1995); each of which is incorporated herein by reference).

A paraptosis-modulating compound can be labeled so as to be detectable using methods well known in the art (Hermanson, supra, 1996; Harlow and Lane, supra, 1988; chap. 9). For example, a paraptosis-modulating compound can be linked to a radioisotope or therapeutic agent by methods well known in the art. A paraptosis-modulating compound that directly binds to an endogenous paraptosis-mediating molecule linked to a radioisotope or other moiety capable of visualization can be useful to diagnose or stage the progression of a clinical stage of a neurodegenerative condition characterized by the organ or tissue-specific presence or absence of a endogenous paraptosis-mediating molecule.

The paraptosis-modulating compounds that inhibit paraptotic cell death taught herein are useful in the invention methods of treating or reducing the severity of a condition associated with excessive cell death, for example, an ischemic condition such as stroke or myocardial infarction; or a neurodegenerative condition. Conversely, a paraptosis-modulating compounds that induce paraptotic cell death are useful in the invention methods of reducing the severity of a condition associated with excessive cell accumulation, for example, a neoplastic condition or an autoimmune condition. A paraptosis-modulating compound can modulate paraptotic activity by binding to an endogenous paraptosis-mediating molecule, to a regulatory molecule that modulates the activity or expression of an endogenous paraptosis-mediating molecule, or to a gene region that controls expression of an endogenous paraptosis-mediating molecule. For example, a paraptosis-modulating compound useful for practicing the claimed invention can be an antibody against a regulator molecule that modulates expression or activity of an endogenous paraptosis-mediating molecule. Alternatively, it may be desired to use populations of random peptide populations to identify further paraptosis-modulating compounds. Those skilled in the art will know or can determine what type of approach and what type of paraptosis-modulating compound is appropriate for practicing the methods of the invention.

The invention therefore provides a method of treating a condition associated with excessive cell accumulation by administering to a subject in need of such treatment an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, where the effective amount of the compound induces paraptotic cell death.

In a separate embodiment, the invention provides a method of treating a condition associated with excessive cell death comprising administering to a subject in need of such treatment an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein the effective amount of the compound inhibits paraptotic cell death.

As used herein, the term “treating” when used in reference to a pathological condition is intended to refer to any detectable beneficial therapeutic effect on the pathological condition of the subject being treated. The beneficial effect can be evidenced, for example, by a delayed onset of clinical symptoms, a reduction in severity of some or all clinical symptoms of the condition, a slower progression of the condition, a reduction in the number of relapses of the condition, a reduction in the number or activity of pathogenic cells, an improvement in the overall health or well-being of the individual, or by other parameters known in the art that are specific to the particular condition.

As used herein, the term “autoimmune condition” refers to a condition characterized by an immune response against the body's own tissues. Autoimmune conditions develop when the immune system destroys normal body tissues caused by a hypersensitivity reaction similar to allergies, where the immune system reacts to a substance that it normally would ignore. The methods described herein for inducing paraptotic cell death can be used to treat a subject where it is desirable to induce cell death in the immunoeffector cells that mediate the autoimmune condition.

As used herein, the term “ischemic condition” refers to a condition in which blood flow is insufficient to support the metabolic demand of a tissue, often due to stenosis or occlusion of a blood vessel. For example, in myocardial ischemia the blood flow to the heart is insufficient to support the metabolic demand of the heart, resulting in myocardial hypoxia and accumulation of waste metabolites, most often due to atherosclerotic disease of the coronary arteries. The term “myocardial infarction,” as used herein, refers to damage to the heart muscle caused by stenosis of one or more of the coronary arteries. The term “stroke,” as used herein refers to a condition that occurs when stenosis of blood vessels carrying oxygen and other nutrients to a specific part of the brain fails to reach that part of the brain such that the brain oxygen supply is cut off resulting in brain damage.

As used herein, the term “neurodegenerative condition” refers to a condition that is characterized by increased or accelerated neural cell death. The methods described herein for inhibiting paraptotic cell death can be used to treat an individual having a condition characterized by a pathologically elevated level of paraptosis, such as occurs in neuronal cells in patients with neurodegenerative conditions, including Parkinson's disease, Huntington's disease, Alzheimer's disease and the encephalopathy that occurs in AIDS patients.

As described herein, paraptosis and apoptosis represent two separate programs of cell death that are induced via distinct molecular pathways, but may be induced simultaneously by a single insult or agent, Consequently, the invention method of treating a condition associated with excessive cell accumulation can be practiced by administering to a subject a combination therapy that consists of an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid to induce paraptotic cell death and an effective-amount of a compound known to induce apoptotic cell death. Compounds known to induce apoptotic cell death are well known in the art and include, for example, doxorubicin, taxol and tamoxifen.

Similarly, the invention method of treating a condition associated with excessive cell death can be practiced by administering to a subject a combination therapy that consists of an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125 to inhibit paraptotic cell death and an effective amount of a compound known to inhibit apoptotic cell death. Compounds known to inhibit apoptotic cell death are well known in the art and include, for example, dominant negative caspase-3 and zVAD.fmk.

A paraptosis-modulating compound of the invention can be useful in the invention methods for treatment of a condition characterized by increased or decreased paraptotic cell death. Various conditions are characterized by an increased or decreased level of paraptosis as compared to the normal level of paraptosis for a particular population of cells. For example, decreased levels of paraptosis are associated with neoplastic conditions, including cancer, where a tumor forms amidst otherwise normal cells in a tissue or organ. In addition, increased levels of paraptosis are associated with a number of neurodegenerative conditions including stroke, trauma, global ischemia, hypoxia, seizure-induced excitotoxicity, and certain neurodegenerative diseases. As set forth herein, the therapeutic methods of the invention are useful for treatment or reduction in severity of conditions associated with either excessive cell accumulation or excessice cell death due to a lack of homeostasis of the paraptotic pathway.

As disclosed herein, paraptosis can be modulated by contacting the appropriate cell or cell population with a paraptosis-modulating compound, for example, a paraptosis-inducing compound such as ceramide, Tumor Necrosis Factor (TNF), caspase-7, caspase-8, α-amino-3-hydroxy-5-methyl-4-isoxazole proprionic acid (AMPA), kainic acid and glutamic acid; or a paraptosis-inhibiting compound such as Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125. Such paraptosis-modulating compounds, therefore, are useful as medicaments for treating a pathology characterized, in part, by aberrant paraptosis. The skilled artisan will recognize the broader usefulness of paraptosis-modulating compounds for therapeutic treatment of conditions characterized by aberrant levels of paraptosis.

Paraptosis can be induced in a cellular system by overexpressing, for example, caspase-7, caspase-8 or caspase-9, the latter in the presence of an apoptosis inhibitor. Caspase-7 and caspase-8 expression induces both apoptotic and paraptotic cell death. Subsequent to the induction of paraptosis, a co-immunoprecipitation assay or similar immunoassay can be performed to identify further compounds that are associated with caspase-9 in paraptotic cells and, consequently, are candidate paraptosis-modulating compound. The paraptosis-modulating activity of the caspase-9-co-immunoprecipitating compound can be verified using the methods known in the art.

The paraptosis-modulating compounds taught herein and useful for practicing the methods of the invention can be formulated and administered by those skilled in the art in a manner and in an amount appropriate for the condition to be treated; the rate of disease progression; severity of symptoms, the weight, gender, age and health of the subject; the biochemical nature, bioactivity, bioavailability and side effects of the particular compound; and in a manner compatible with concurrent treatment regimens. An appropriate amount and formulation for decreasing the severity of a neoplastic condition, autoimmune condition, ischemic condition, neurodegenerative condition in humans can be extrapolated from credible animal models known in the art of the particular condition. It is understood, that the dosage of a paraptosis-modulating compound may have to be adjusted based on the binding affinity of the paraptosis-modulating compound for a second compound, such that a lower dose of a paraptosis-modulating compound exhibiting significantly higher binding affinity can be administered compared to the dosage necessary for a paraptosis-modulating compound with lower binding affinity.

The total amount of a paraptosis-modulating compound can be administered as a single dose or by infusion over a relatively short period of time, or can be administered in multiple doses administered over a more prolonged period of time. Such considerations will depend on a variety of case-specific factors such as, for example, in case of a neurodegenerative disease it will depend on whether the disease category is characterized by acute episodes or gradual deterioration. For example, for a subject affected with chronic deterioration the paraptosis-modulating compound can be administered in a slow-release matrix, which can be implanted for systemic delivery or at the site of the target tissue. Contemplated matrices useful for controlled release of therapeutic compounds are well known in the art, and include materials such as DepoFoam™, biopolymers, micropumps, and the like.

The paraptosis-modulating compounds can administered to the subject by any number of routes known in the art including, for example, systemically, such as intravenously or intraarterially. A paraptosis-modulating compound can be provided, for example, in the form of isolated and substantially purified polypetides and polypeptide fragments in pharmaceutically acceptable formulations using formulation methods known to those of ordinary skill in the art. These formulations can be administered by standard routes, including, for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. In addition, a paraptosis-modulating compound can be incorporated into biodegradable polymers allowing for sustained release of the compound useful for reducing the severity of a neoplastic condition or neurodegenerative condition. Biodegradable polymers and their use are described, for example, in Brem et al., J. Neurosurg. 74:441-446 (1991), which is incorporated herein by reference.

A paraptosis-modulating compound can be administered as a solution or suspension together with a pharmaceutically acceptable medium. Such a pharmaceutically acceptable medium can be, for example, sterile aqueous solvents such as sodium phosphate buffer, phosphate buffered saline, normal saline or Ringer's solution or other physiologically buffered saline, or other solvent or vehicle such as a glycol, glycerol, an oil such as olive oil or an injectable organic ester. A pharmaceutically acceptable medium can additionally contain physiologically acceptable compounds that act, for example, stabilize the paraptosis-modulating compound, increase its solubility, or increase its absorption. Such physiologically acceptable compounds include, for example, carbohydrates such as glucose, sucrose or dextrans; antioxidants such as ascorbic acid or glutathione; receptor mediated permeabilizers, which can be used to increase permeability of the blood-brain barrier; chelating agents such as EDTA, which disrupts microbial membranes; divalent metal ions such as calcium or magnesium; low molecular weight proteins; lipids or liposomes; or other stabilizers or excipients. Those skilled in the art understand that the choice of a pharmaceutically acceptable carrier depends on the route of administration of the compound containing the paraptosis-modulating compound and on its particular physical and chemical characteristics.

Formulations suitable for parenteral administration include aqueous and non-aqueous sterile injection solutions such as the pharmaceutically acceptable mediums described above. The solutions can additionally contain, for example, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

For applications that require the compounds and compositions to cross the blood-brain barrier, formulations that increase the lipophilicity of the compound are particularly desirable. For example, the paraptosis-modulating compound can be incorporated into liposomes (Gregoriadis, Liposome Technolosy, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer.

A paraptosis-modulating compound can also be prepared as nanoparticles. Adsorbing peptide compounds onto the surface of nanoparticles has proven effective in delivering peptide drugs to the brain (see Kreuter et al., Brain Res. 674:171-174 (1995)). Exemplary nanoparticles are colloidal polymer particles of poly-butylcyanoacrylate with paraptosis-modulating compound adsorbed onto the surface and then coated with polysorbate 80.

Image-guided ultrasound delivery of a paraptosis-modulating compound through the blood-brain barrier to selected locations in the brain can be utilized as described in U.S. Pat. No. 5,752,515. Briefly, to deliver a paraptosis-modulating compound past the blood-brain barrier a selected location in the brain is targeted and ultrasound used to induce a change detectable by imaging in the central nervous system (CNS) tissues and/or fluids at that location. At least a portion of the brain in the vicinity of the selected location is imaged, for example, via magnetic resonance imaging (MRI), to confirm the location of the change. An paraptosis-modulating compound in the patient's bloodstream can delivered to the confirmed location by applying ultrasound to effect opening of the blood-brain barrier at that location and, thereby, to induce uptake of the paraptosis-modulating compound.

In addition, polypeptides called receptor mediated permeabilizers (RMP) can be used to increase the permeability of the blood-brain barrier to molecules such as therapeutic agents or diagnostic agents as described in U.S. Pat. Nos. 5,268,164; 5,506,206; and 5,686,416. These receptor mediated permeabilizers can be intravenously co-administered to a host with molecules whose desired destination is the cerebrospinal fluid compartment of the brain. The permeabilizer polypeptides or conformational analogues thereof allow therapeutic agents to penetrate the blood-brain barrier and arrive at their target destination.

In current treatment regimes for neoplastic conditions, autoimmune conditions, ischemic conditions as well as for neurodegenerative conditions, more than one compound is often administered to an individual for management of the same or different aspects of the disease. Similarly, in the methods of the invention a paraptosis-modulating compound can advantageously be formulated with a second therapeutic compound such as an apoptosis-modulating compound, an anti-inflammatory compound, an immunosuppressive compound or any other compound that manages the same or different aspects of the disease. Such compounds include, for example, methylprednisolone acetate, dexamethasone and betamethasone. Contemplated methods of treating or reducing the severity of a neoplastic conditions, autoimmune conditions, ischemic conditions as well as for neurodegenerative conditions, include administering a paraptosis-modulating compound alone, in combination with, or in sequence with, such other compounds. Alternatively, combination therapies can consist of fusion proteins, where the paraptosis-modulating compound is linked to a heterologous protein, such as a therapeutic protein.

All journal article, reference, and patent citations provided above, in parentheses or otherwise, whether expressly stated or not, are incorporated herein by reference in their entirety.

It is understood that modifications which do not substantially affect the activity of the various embodiments of this invention are also included within the definition of the invention provided herein. Accordingly, the following examples are intended to illustrate but not limit the present invention.

### EXAMPLE I

### Inducers of Paraptosis

This example demonstrates the identification of agents capable of inducing paraptotic cell death.

Human embryonic kidney 293T cell line cells were used to assay for inducers and inhibitors of paraptosis. These cells were cultured in high glucose DMEM (Life Technologies) supplemented with 10% FBS (Sigma) and 1 penicillin/streptomycin (Life Technologies). The cultures were incubated at 37° C. in 95% air 5% carbon dioxide with 95% humidity.

Rat neuronal primary cell cultures were also used to assay for inducers and inhibitors of paraptosis. Briefly, primary striatal, cortical and hippocampal cultures were prepared from 17-day-old Sprague-Dawley rat embryos (B&K). The tissue was dissected, minced and trypsinized for five minutes using 0.25% trypsin (Cell Grow). After the addition of 10% horse serum (Life Technologies) to inhibit the trypsin, the cell suspension was triturated 15-20 times with a 10 ml syringe and centrifuged for five minutes at 800×g. The pellet was resuspended in MEM-PAK (UCSF Cell Culture facility), supplemented with 2.02 μm glucose, 2 mM GlutaMax (Life Technologies) and penicillin/streptomycin (100 U/ml). The suspension was filtered through a 70 μm cell strainer and the final culture medium contained 5% horse serum. Subsequently, 3-4×10⁵ cells per cm² were seeded onto either poly-D-lysine precoated 8-well chamber slides (Becton-Dickson Labware) or 96 well plates precoated with 50 μg/ml of poly-D-lysine (Sigma) in water. After 30 minutes of incubation time, unattached cells were removed together with the medium and replaced with glucose enriched MEM-PAK plus 5% horse serum. The cultures were then incubated at 37° C. in 95% air 5% carbon dioxide with 95% humidity. Cultures were used for experiments between day 1 and day 7 when glial contamination was at a minimum.

Subsequently, compounds were added to 293T cells and/or rat neuronal primary cell culture cells in order to determine if the compounds could induce paraptosis. Cell death was assayed using the lactate dehydrogenase (LDH) assay, in which the relative amount of enzyme released from dying cells in the medium is measured by following the disappearance of nicotinamide adenine dinucleotide, reduced form (NADH) in the following reaction: pyruvate+NADH<−>NAD⁺+lactate. Briefly, 50 μl of culture medium for either 293T cells or 100 μl of culture medium for neuronal primary culture were dispensed in a 96 well plate to which 100 μl of a solution consisting of 5 mg of NADH dissolved in 20 ml of PBS and 0.5 ml of 100 mM NaPyruvate. NADH disappearance was assessed by kinetic photmetric readings at a 340 nm wavelength at 19 minute intervals over a period of 2 hours and 30 minutes.

The following compounds were found to induce paraptosis when used at the concentrations indicated below:

1. C2 ceramide (N-Acetyl-D-sphingosine) used at 15-100 μM in both 293T cells and rat neuronal primary culture cells.

2. Tumor Necrosis Factor-α used at 1-10 ng/ml in 293T cells.

3. AMPA (alpha-amino-3-hydroxy-5-methyl-4-isoxazolepropionic acid) at 35-500 μM in rat neuronal primary cell cultures.

4. Kainic Acid (2-Carboxy-3-carboxymethyl-4-isopropenylpyrrolidine) at 35-500 μM in rat neuronal primary cell cultures.

5. Glutamic Acid ((S)-2-aminopentanedioic acid) at 50-500 μM in rat neuronal primary cell cultures.

In addition, procaspase-7 and procaspase-8 were also able to induce paraptosis when expressed in 293T cells. Constructs of pcDNA3 containing procaspase-7 or procaspase-8 were transiently transfected into 293T cells using Lipofect-Amine (GIBCO-BRL) according to the manufacturer's instructions. Briefly, 1×10⁶ 293T cells were seeded in 6 cm dishes, and transfected the next day using a ratio of DNA:Lipofect-Amine of 1 μg:5 μl. Transfection efficiency was 60-80% for 293T cells, as determined by X-gal staining after transfection of a β-galactosidase construct.

### EXAMPLE II

### Inhibitors of Paraptosis

This example demonstrates the identification of agents capable of inhibiting paraptotic cell death.

Paraptosis was induced in 293T cells by expressing the intracellular domain of the insulin-like growth factor I receptor (IGFIR-IC) in these cells using transient transfection as described above.

Test compounds were added to IGFIR-IC transfected 293T cells at the time of transfection. The copper chelator, 1-10-phenantroline (ortho-phenantroline) was able to inhibit IGFIR-IC induced paraptosis when added to transfected 293T cells at a concentration of 50 nM. In addition, H89, an inhibitor of protein kinase A, was able to inhibit IGFIR-IC induced paraptosis when added at a concentration of 10-20 μM. Furthermore, 1-10-phenantroline was able to inhibit paraptosis induced by caspase 8 or caspase 9.

In addition, AIP-1 (Alg-2 interacting protein 1) and a dominant negative mutant of TRAF2 were able to inhibit IGFIR-IC induced paraptosis when co-transfected with the IGFIR-IC construct. The TRAF2 dominant negative mutant contains a deletion of the amino terminal 271 amino acids of the protein. The ratio of IGFIR-IC:AIP1 and IGFIR-IC:TRAF2DN was 1:3. Furthermore, antisense oligonucleotide constructs for JNK1 or JNK2 were able to inhibit IGFIR-IC induced paraptosis in 293T cells. Antisense oligonucleotide constructs for JNK1 or JNK2 (50-100 nM) were transfected into 293T cells one day before transfection with the IGFIR-IC construct. For all inhibition assays, cell viability was measured using the LDH assay.

We claim:

1. A method of inducing paraptotic cell death in a cell comprising contacting said cell with an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, wherein said effective amount of said compound induces paraptotic death of said cell.

2. The method of claim 1, wherein said paraptotic cell death is induced in a mammal.

3. The method of claim 2, wherein said mammal is a human.

4. A method of inhibiting paraptotic cell death in a cell comprising contacting said cell with an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein said effective amount of said compound inhibits paraptotic death of said cell.

5. The method of claim 4, wherein said paraptotic cell death is inhibited in a mammal.

6. The method of claim 5, wherein said mammal is a human.

7. A method of treating a condition associated with excessive cell accumulation comprising administering to a subject in need of such treatment an effective amount of a compound selected from the group consisting of ceramide, Tumor Necrosis Factor (TNF), caspase-7, caspase-8, α-amino-3-hydroxy-5-methyl-4-isoxazole proprionic acid (AMPA), kainic acid and glutamic acid, wherein said effective amount of said compound induces paraptotic cell death.

8. The method of claim 7, wherein said compound is part of a combination therapy that further comprises an effective amount of a compound known to induce apoptotic cell death.

9. The method of claim 7 or 8, wherein said condition is a neoplastic condition.

10. The method of claim 7 or 8, wherein said condition is an autoimmune condition.

11. A method of treating a condition associated with excessive cell death comprising administering to a subject in need of such treatment an effective amount of a compound selected from the group consisting of Alg-2-interacting protein 1 (AIP-1), Jun N-terminal kinase 1 (JNK1) neutralizing agent, Jun N-terminal kinase 2 (JNK2) neutralizing agent, TNF Receptor-Associated Factor 2 (TRAF2) neutralizing agent, ortho-phenanthroline and the JNK inhibitor SP 600125, wherein said effective amount of said compound inhibits paraptotic cell death.

12. The method of claim 11, wherein said compound is part of a combination therapy that further comprises an effective amount of a compound known to inhibit apoptotic cell death.

13. The method of claim 11 or 12, wherein said condition is an ischemic condition.

14. The method of claim 13, wherein said ischemic condition is a stroke.

15. The method of claim 13, wherein said ischemic condition is a myocardial infarction.

16. The method of claim 11 or 12, wherein said condition is a neurodegenerative condition.
